(19) United States
(12) Patent Application Publication
(10) Pub. No.: US 2008/0269629 A1
(43) Pub. Date: Oct. 30, 2008

(54) Multimodal therapeutic and feedback system

(76) Inventor: Robert Howard Reiner, Bedford, NY (US)

Correspondence Address: GREENBERG TRAURIG, LLP, MET LIFE BUILDING, 200 PARK AVENUE, NEW YORK, NY, 10166, US

(21) Appl. No.: 11/789,472

(22) Filed: Apr. 25, 2007

(51) Int. Cl. A61B 5/0482

(52) U.S. Cl. 600/544

(57) ABSTRACT

The generation of alpha waves in a mammalian subject may be stimulated through a process of measuring a wave-from produced by the electrical activity of the subject's brain, analyzing the wave-form to determine changes in its frequency, and delivering audio and visual stimulation to the subject, each at a rate selected to vary the frequency of the wave-form until any alpha state is achieved. The rate may be selected by the subject, by a therapist, or may be varied automatically through a feedback mechanism.

### TECHNICAL FIELD

The present invention is related to the field of methods and apparatus for reducing anxiety and promoting relaxation in mammalian subjects.

### BACKGROUND

There is now ample evidence that many psychiatric and medical disorders are characterized physiologically by increased (and exaggerated) arousal of the sympathetic branch of the autonomic nervous system (ANS). This imbalance, often described as the body's fight or flight response typically stems from elevated arousal in the Sympathetic Nervous System (SNS) and decreased arousal in the parasympathetic nervous system (PSNS), which otherwise is associated with relaxation. For the most part, the introduction of a stressor will increase activity in the SNS or the ratio of SNS activity to PSNS activity, while the introduction of methods that induce relaxation will increase activity in the PSNS.

An effective strategy known to reliably increase PSNS activity and the relaxation response is slow diaphragmatic breathing. The typical respiration rate needed to increase PSNS activity is between 4-9 breaths per minute, though this varies depending on the individual. Slow diaphragmatic breathing is known to generate relaxation when the heart rate increases during inhalation and decreases during exhalation in a consistent manner. As the difference in inhalation and exhalation heart rates increases (swings), greater levels of relaxation are observed. This temporary disabling of the “fight or flight” response is called respiratory sinus arrhythmia (RSA). It has been found that this desired state of relaxation can be achieved when respiration is reduced to approximately six breaths per minute and originates almost exclusively from the diaphragm (i.e., from the belly rather than the chest). It is no coincidence that the overwhelming majority of relaxation techniques (e.g. yoga, meditation, etc.) include breathing retraining and mindfulness as central components. Various forms of breathing retraining and mindfulness have been found to be effective treatments and/or treatment adjuncts for anxiety disorders and other disorders of autonomic dysregulation.

While paced deep rhythmic breathing has been shown to be quite effective as a strategy to reliably increase parasympathetic arousal, there are several limitations to its successful implementation. Primarily, without proper physiological assessments, there is no way to ensure that one is maximizing his relaxation response. Biofeedback techniques that directly measure autonomic functioning have been found to combat this difficulty because they provide direct feedback, completing a loop that nature did not build in. Such techniques rely on the monitoring of physiological parameters that are known to correlate with SNS or PSNS arousal, such as heart rate variability (HRV), galvanic skin resistance (GSR), peripheral skin temperature, and muscle tension (EMG). Another technique is to have the subject report on his own sense of well-being (Self Report) and to adjust his breathing rate accordingly. The completion of this loop provides an opportunity to learn through self correction and, eventually, alteration of physiological state, in real time.

HRV is the most reliable and direct measure of PSNS arousal. In a mammalian subject, heart rate increases with every breath taken and decreases with every breath exhaled. Typically, the heart rate of a normally relaxed human subject will vary by some 10 to 20 beats per second from the subject's resting heart rate. When the subject is in an anxious state, the variation may be limited to about 5 beats per minute. Increases in PSNS activity can be tracked by the increasing variability of the heart rate. The actual degree of heart rate variability that is desired will differ depending on the age, size/weight, cardiovascular health, breed, and species of the mammalian subject being considered.

Brain wave stimulation may also be used to promote relaxation. Currently, there are two different methodologies or vehicles that determine the quality, quantity, timing, and strength of the neurological intervention. Brain wave frequencies, which are measured by an instrument called an electroencephalograph (EEG), have been categorized into four major groups. The slowest waves, typically generated during deep sleep, are known as delta waves, and are associated with frequencies dominating in the range of 1 to 4 cycles per second. Theta waves have frequencies in the range of 5 to 8 cycles per second and are linked with decreased awareness of the physical world, daydreaming, sluggishness, depression, irritability, dreamless and/or light or restless sleep, and attention deficit disorder (ADD). Alpha waves, generated in the range of 9 to 13 cycles per second, are linked to a feeling of alertness but not with active processing of information. The goal of experienced meditators is to achieve an alpha state, which is typically associated with contentment and satisfaction. Beta states, starting at approximately 14 cycles per second, are associated with active processing of information, high states of alertness and extroversion, hyper vigilance and anxiety. Audio-Visual Entrainment (AVE), facilitates the attainment of desirable neurological states, such as alpha by producing soft tones and blinking off-white lights, via headphones and special headsets programmed at the desired frequencies (about 9 to 14 cycles per second). AVE can be also be used to achieve the other neurological states (i.e., delta, theta and beta) by modifying the frequencies at which the pulses of light and/or sound are delivered.

A significant difference between audio-visual stimulation and conventional biofeedback is the mode of delivery. The use of audio-visual entrainment is similar to a patient taking medication, in that a calibrated dosage is offered by the doctor and absorbed by the patient. For the most part, the intervention is not at all contingent on the patient's behavior. In biofeedback, the opposite holds true. The patient takes an active role in this intervention because he will receive nothing unless he produces the expected physiological response. Biofeedback techniques have long been used as a clinically effective method for increasing awareness and improving levels of physiological functioning.

Electroencephalographic biofeedback (“EEG biofeedback”) uses computerized electronic measurement devices placed on the surface of the head to monitor brain wave activity. The computer “feeds back” to the subject important information relevant to desired neurological state. The feedback loop is generally closed by the subject observing a tracing of his brain wave patterns, and modifying the patterns until the desired neurological state is achieved. Through guided techniques the subject is able to learn to significantly increase brain waves (beta) which are compatible with stronger attentional focus and enhanced mental performance. The biofeedback technique facilitates learning to create desirable brain waves automatically, a skill which quickly generalizes to everyday life without the intervention of biofeedback. EEG biofeedback has physiological effects similar to those created by medication, but has no reported adverse side effects, is painless, and often provides long lasting results.

Therapeutic massage provides another approach to increase PSNS activity and promote relaxation. In one approach, the subject receives a rolling massage (i.e., an even pressure is exerted continuously) moving between the upper and lower portions of the back. Benefits of massage include stimulation of the PSNS, resulting in reduction of heart rate, muscle tension, blood pressure, and vascular stiffening. Furthermore, therapeutic massage is believed to generate endorphins which are released into the bloodstream. These neurotransmitters have pain-relieving properties, reduce stress, and bolster the immune system. The stress reducing properties of therapeutic massage have received consistent support in a review of the literature.

### SUMMARY

The present disclosure describes a combination of the aforementioned techniques into a system of therapy to stimulate the PSNS to a degree that exceeds that expected from the use of single or paired techniques. The combination of these procedures causes the mammalian brain to generate deep relaxation, reduce both physiological and psychological stress and promote cognitive clarity. Besides promoting relaxation and relief of anxiety, the invention may be used to promote alpha-wave states and to prime subjects for hypnosis.

One embodiment comprises a system including a means for providing massage, means for providing pulses of light, and means for generating pulses of audible sounds. A controller is optionally provided to control the rate at which the massage element is moved. Controllers may also be provided to vary the rate at which the light and sound pulses are delivered, as well as their intensity. Biofeedback devices may be included to provide signals to the controllers, so that the rates and intensities of the massaging motions, light pulses and sound pulses may be varied in response to one or more of the subject's physiological parameters.

Another embodiment comprises a method of promoting relaxation and relief from anxiety in a mammalian subject by delivering audio and visual stimulation to the subject, and massaging the subject in a cyclic movement or providing other physical stimuli so that the subject is induced to coordinate its breathing with the cyclic movement of the applied massage or stimuli so as to achieve RSA. In a related embodiment, audio and visual stimulation may be delivered as pulses of light to one or both of the right and left visual fields of the subject's eyes, and as pulses of sound to the subject's ears. Audio and visual stimulation may be started after the controlled breathing exercise is underway, after RSA has been achieved, or at the same time as the initiation of the breathing exercise. In an alternative, the audio and visual stimulation may be started before the controlled breathing exercise has begun. In variations of these embodiments, biofeedback control techniques may be used to optimize the rates at which the massage, light pulses and sound pulses are delivered, as well as their intensities. Yet another embodiment comprises the combination of AVE with biofeedback to further enhance the subject's ability to generate, and realize the benefits of, the alpha state.

### BRIEF DESCRIPTION OF THE DRAWINGS

FIG. 1 is a profile view of a human subject in a system according to one embodiment, including a cutaway view of the reclining chair component.

FIG. 2 is a profile view of a human subject in a second embodiment.

### DETAILED DESCRIPTION

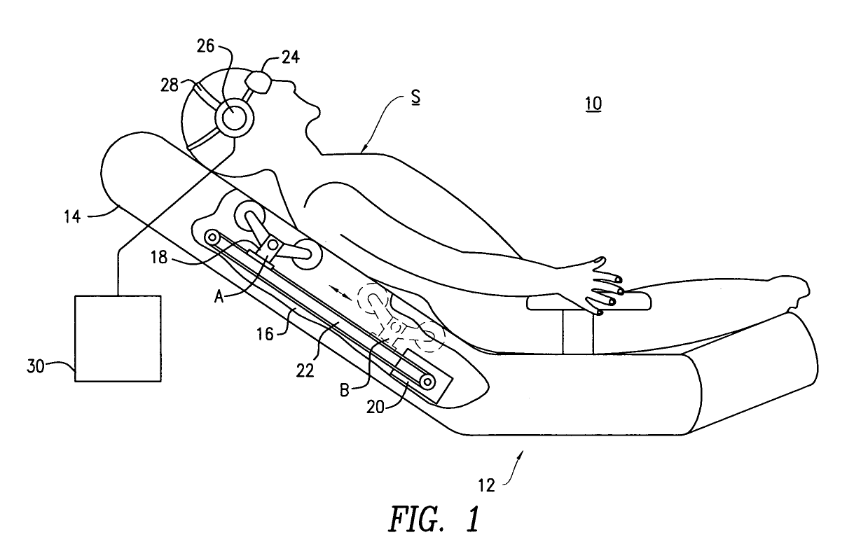

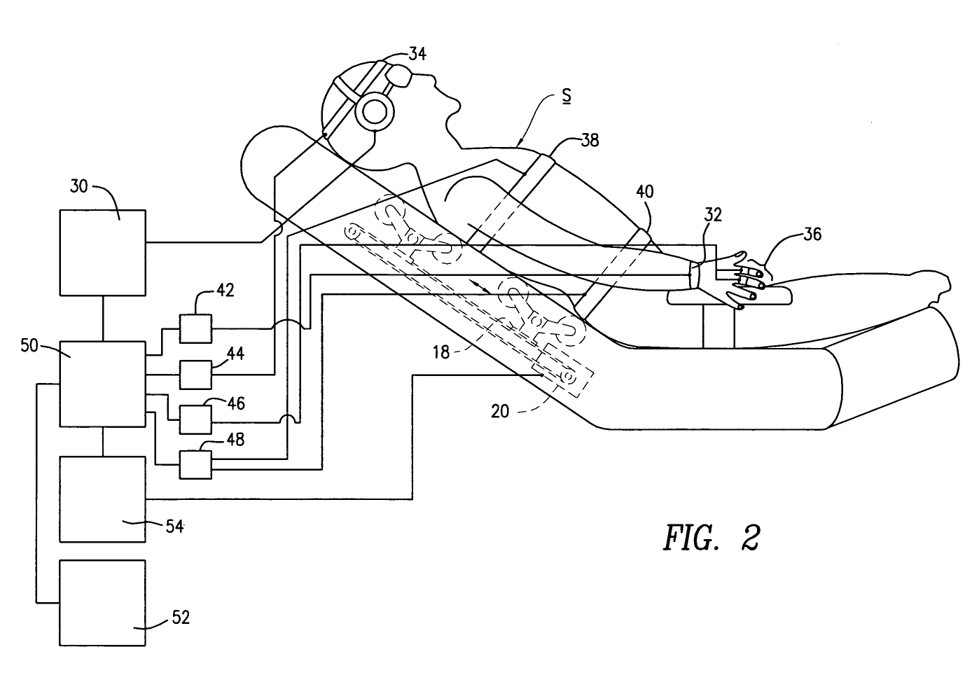

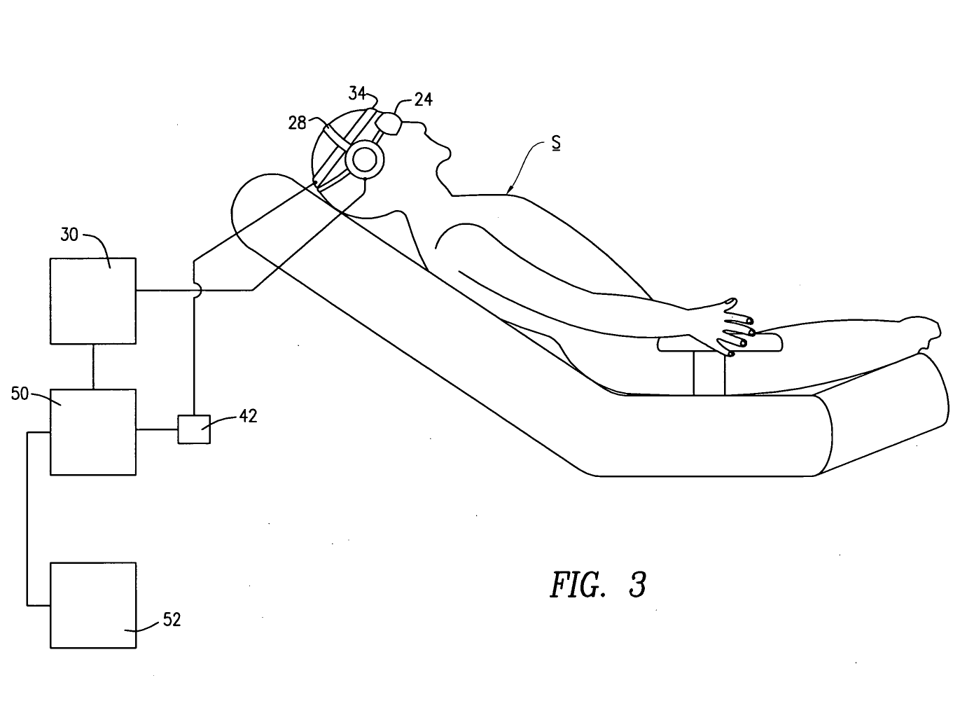

FIGS. 1 and 2 illustrate embodiments of a multimodal therapeutic system 10. To facilitate the description of the embodiments, the figure includes a depiction of a human subject S, who is not part of the system itself. Details of the subject, such as eyes, ears, etc., are also referred to herein, but are not shown in the figure. The subject may also be a non-human mammalian subject.

Turning to FIG. 1, subject S preferably lies recumbent in a chair 12 having a reclining back 14, and a roller system 16, preferably incorporated within the chair back 14. The roller system 16 comprises a set of rollers or other massage element for exerting pressure 18, an actuator comprising a motor 20 and a mechanical linkage 22 operationally connected to the set of rollers 18 so that the set of rollers 18 can be moved in a continuous or patterned reciprocating motion between location A, proximal to the subject's upper back, and position B proximal to the subject's lower back. As discussed elsewhere in this disclosure, massage may be applied by, without limitation, using massage elements other than rollers, or directly by the care-giver. It may also be desirable to apply the massage to other parts of the subject's body other than the subject's back.

The system 10 further preferably comprises a set of glasses or goggles 24 which have a number of bulbs, light-emitting diodes, or other light-producing material now known or to become known, arranged to direct pulses of light alternately, selectively, or simultaneously to the right and left visual fields of the subject's eyes. The element 24 may also be configured as ambient or other light emitting fixtures placed proximate the subject to appear visible within the subject's field of vision and need not be worn by the subject. The light applied can be varied as to color and/or intensity to suit the desired result over a varied subject population.

The system 10 also preferably comprises a pair of sound transducers such as acoustic speakers 26, or other sound generating or emitting devices that are known or which may become known, which, in this embodiment, are attached to a headset 28 worn on the subject's head, and deliver pulses of sound to the subject's ears. Speakers 26 may also be positioned proximate the subject within the subject's range of hearing, or be affixed to or part of the chair, and need not be worn by the subject. A controller 30 is optionally provided to coordinate or vary the timing and intensity of the light pulses and sound pulses, and/or, as described further herein, the rate of movement of the rollers 18.

The embodiment of FIG. 2 includes elements similar to those illustrated in FIG. 1, indicated by the same reference numbers, and further includes sensors and monitors that would be useful in monitoring the subject's physiological parameters, preferably to be utilized in one or more biofeedback loops. Such biofeedback loops can be used to generate control signals by which selected components of said system 10 can be controlled to optimize the subject's physical state and/or state of relaxation. Sensors may include, by way of non-limiting example, one or more of the following: (1) a strain gauge or other type of blood pressure detector with or without a heart rate detector 32 for detecting changes in blood pressure and/or heart rate, (2) electrodes 34 to detect electrical current generated by the brain, (3) electrodes 36 for measuring galvanic skin resistance, or (4) strain gauges in elastic belts 38, 40 surrounding the subject's chest and abdomen for monitoring the subject's breathing pattern.

Electronic monitors, each including an electrical measurement circuit (not shown) and a signal converter (not shown) of types known in the art are provided to measure changes in the sensors and convert them to signals for controlling system components and/or for displaying results to a care-giver and/or the subject. The monitors, respective to the order of measuring elements referenced above, may include a blood pressure and/or heart rate monitor 42, an electroencephalograph 44, a galvanic skin resistance monitor 46, and/or a breathing monitor 48. In variations of the embodiment, an electrical measurement circuit and/or signal converter may be housed with a sensor as a single unit.

Physiological parameters that are not mentioned above, but which are relevant to the relaxed state and brain wave entrainment of a subject, such as skin temperature or muscle tension, as well as the sensors and monitors useful for monitoring such parameters, will be apparent to those having ordinary knowledge of the physiology of anxiety and its reduction in mammalian subjects.

Turning back to FIG. 2, the monitors 42-48 may be arranged to provide output signals to a microprocessor 50 for additional processing and/or for display on a single visual monitor 52. Further, the microprocessor may be arranged to provide signals to the aforementioned controller 30 to vary the rates and/or intensities of the pulses of light or sound, or to a motor controller 54 to vary the rate at which the rollers 18 are moved. Such variations would be made to optimize the relaxed state of the subject, and may be based upon signals from the aforementioned monitors, the intervention of the care-giver, self-monitoring by the subject, or some combination of the above.

Embodiments also include methods for promoting relaxation and relief from anxiety in a human or mammalian subject. Such methods combine the techniques of controlled diaphragmatic breathing to achieve respiratory sinus arrhythmia (RSA), audiovisual entrainment (AVE) and therapeutic massage, each of which is discussed briefly in the Background section, above.

With reference to FIG. 1, in a method of promoting relaxation, the human subject S lies recumbent in the chair 12, with goggles 24 and headset 28 in place. The roller system 16 moves the rollers 18 up and down the subject's spine at a pre-selected rate, preferably in the range of 4 to 9 times per minute for a human subject, thus massaging the subject's back. Other rates of massage are also contemplated in this embodiment, the selection of which will depend on the individual's response. Yet other rates of massage may be used for non-human subjects, depending on the species and breed of mammal. Other parameters that would influence the desired massage rate would include, without limitation, the size, weight, age, and cardiovascular health of the subject. During this massaging step, the subject is induced to practice controlled diaphragmatic breathing, preferably breathing in as the rollers move upward to the upper portion of the subject's back and breathing out as the rollers move downward to the lower portion of the subject's back, although other reactions to roller speed and position are possible.

During, prior or subsequent to a time when RSA has been achieved, the goggles deliver pulses of light separately, simultaneously or alternately, to either or both of the right and left visual fields of the subject's eyes, preferably at a rate in the range of about 10 to 20 pulses per second, depending on the subject's reactions. Experience has shown that desirable results are often obtained at a rate of 14 cycles per second, which is also the target rate frequently sought by experienced meditators. Pulses of soft white light are generally preferred, although other colors, such as orange, brown or blue may be used, depending on the preference or reactions of the subject. It is known, however, that certain subjects having seizure disorders should not be exposed to pulsating lights. For such persons, the step of delivering pulsating lights to the subject's eyes may be eliminated.

During, prior or subsequent to a time when RSA has been achieved and/or the light pulses being applied, pulses of sound (e.g. white or pink noise or tones of selected frequency or multi-frequency tones) are delivered through the speakers 26 in the headset 28, preferably at a rate in the range of about 10 to 20 pulses per second. In one embodiment, pulses of light and pulses of sound are synchronized at a rate of about 14 pulses per second, although the respective pulses may be delivered asynchronously.

As presently understood, in mammals such as humans, successful audio and visual stimulation will cause the same effects as deep meditation, with brain waves synchronized to the light and sound pulses at a frequency in the range of about 10 to 20 pulses per second. Optimum results are often achieved at a frequency of about 14 pulses per second, with a breathing rate of 6 breaths per minute. In variations of the embodiments herein, audio and visual stimulation may be initiated prior to, at the same time as, or subsequent to the movement of the roller or other pressure applying device.

In other embodiments the subject can be monitored by a number of techniques now known or to become known. Among the simplest, for example, is for a person administering the treatment to observe the subject's breathing to ensure that the subject is breathing from the diaphragm rather than from the chest and is inhaling and exhaling in synchrony with movement of the rollers 18. Other methods of monitoring breathing include mechanical means, one of which is the use of strain gauges incorporated into one or more elastic belts 38, 40 that are secured around the chest and/or abdomen of the subject, although pressure sensors at the nostrils may also be used. Such strain gauges can be used to monitor the rates of inhalation and exhalation, as well as the desired breathing form (i.e., diaphragmatic breathing). The monitored results may be displayed to the care-giver who can guide the subject to breathe properly, or to a visual or other perceptible cue delivered to the subject for self-regulation, or fed to a control mechanism for automated adjustment of system parameters.

The effectiveness of the relaxation method can be monitored by a number of methods, including, for example, self-reporting by the subject on his or her physical and emotional states. More objective monitoring can be performed by monitoring and/or measuring physiological parameters that are indicative of SNS or PSNS arousal, such as heart rate variability or galvanic skin resistance. The results of such monitoring can be viewed by the care-giver, who can then adjust the rate at which the pressure to the back (or other body part) is applied or the rates or intensities of the pulses of light or sound to effectuate a more relaxed state in the subject, or such adjustments may be made by the subject, or combinations of the aforementioned. Alternatively, the physiological monitors may be connected to a microprocessor or other computing device or controller 50 which would, in turn, send signals to controllers, such as controllers 30 and 54, to adjust the execution of the treatment method to further increase the relaxed state of the subject in an automated fashion, without the intervention by the care-giver or the subject.

The disclosures made herein support yet another method of achieving the relaxed state associated with PSNS stimulation. As discussed in the Background section, each of the interventions of AVE and biofeedback have been shown to be successful in aiding a subject to achieve a relaxed state. As noted therein, the role of the subject in biofeedback is far more active than its role in accepting AVE alone. That is, with biofeedback, the patient must generate the desired response (e.g., the alpha state) before the reward (e.g., a state of relaxed alertness) is delivered.

To date, there have been no documented scientific attempts made to combine AVE with biofeedback. Without limiting the disclosure by theory, it is hypothesized that deep states of relaxation are achieved using the alpha waves generated by the AVE process as reinforcement during feedback sessions. The mechanism of such reinforcement is that, when the patient generates alpha waves during feedback sessions, the reward will be alpha waves generated by AVE. Paradoxically, as the subject learns to create increasing amounts of alpha waves, its pleasure will be enhanced because the reward will also be the positive reinforcement of alpha wave entrainment through AVE. Furthermore, through positive reinforcement, the subject learns an important meditative skill that will enable the subject to generate desirable amounts of alpha waves without the intervention of AVE or biofeedback.

A non-limiting embodiment of a process for achieving such positive reinforcement can be illustrated with reference to features referenced in FIG. 3. Therein, the subject S is equipped with the goggles 24 and headset 28 of the AVE system, which delivers light and sound pulses regulated by controller 30. As discussed with respect to the embodiments related to FIG. 2, various means of delivering pulses of light to the subject's eyes may be used, other than the goggles 24. Similarly, means of delivering sound to the subject's ears may be used, other than the headset 28. Neither the light-delivery means nor the sound-generating means need to be worn on the subject's head. Returning to FIG. 3, the subject S is further provided with electrodes 34 to connected to a device, of a type now known or to become known, used to measure the electrical activity of the brain. Such measurements are monitored by electroencephalograph 42, which provides an output signal to microprocessor 50. The device used to measure and/or monitor the electrical activity of the brain are not necessarily limited to the electrodes 34 and electroencephalograph 42, as long as such device is capable of detecting and monitoring brain wave frequencies in the desired range. Returning, again, to FIG. 3, the microprocessor 50 processes the output signal of the electroencephalograph 42, or other suitable monitoring device, and delivers it to a visual display 52 which the subject observes to monitor the frequency of its brain waves. To complete the feedback loop, the controller 30 can then be manipulated by the subject or an observer to vary the frequency and/or intensity of the pulses of light and sound delivered, respectively, by the goggles 24 and headset 28, or other appropriate light and sound generating means. Alternatively, microprocessor 50 may deliver a signal to controller 30 to vary the frequency and/or intensity of the pulses of light and/or sound, thus completing the feedback loop without outside intervention, automatically varying the light and sound in a manner that induces the subject's brain waves to alter to or remain at a desired frequency indicative of a desired mental state.

It should be understood that the embodiments discussed herein are merely exemplary and that a person skilled in the relevant arts may make many variations and modifications without departing from the spirit and scope of the invention. Also, elements described in certain embodiments may be used alone or in combination or as alternatives to elements described in other embodiments. The treatment, for example, may be administered in fully automated fashion, or fully manual fashion, or in some combinations thereof, with intervention permitted (or not permitted) by the subject, care-giver, system controller or combinations thereof.

By way of further example, with respect to the system 10, it may be desired to incorporate various components into the chair, such as the speakers 26, the controllers 30 and 54, or the monitors 42-48 and microprocessor 50.

With respect to the massage element, it may be desired to apply pressure to the subject by means other than a roller. Non-limiting examples of such means include manual application, pistons, eccentric cams, pneumatic or hydraulic bladders, or vibratory elements. Means of providing motive force to such massage elements may include a mechanical actuator, not limited to the combination of motor 20 and mechanical linkage 22 that has already been described herein, or means incorporating pneumatic or hydraulic actuators, or other actuators that are known or may become known. In further variations, it may be desirable to have the massage applied directly by the care-giver, in which instance the massage element may be a portion of the care-giver's body, such as the hands, and the actuator would be the care-giver's muscles. Massage could also be applied to parts of the subject's body other than the back.

Also, while in embodiments it is contemplated that controlled or preferred rates of breathing be induced by applied pressure such as massage, other physical stimuli can be utilized to induce the desired breathing pattern, such as pressure cues to other parts of the subjects body, or by auditory instruction, or changes in position (e.g. by pitching motion of an object on which the subject is located). In one further non-limiting example, pressure can be applied to and/or removed from the abdomen proximate the diaphragm of the subject to provide the desired breathing cues.

By way of further non-limiting examples of embodiments, the light pulses of one or varied or varying color and/or intensity may be delivered to the right and left eyes synchronously or in alternation, or may be delivered at different rates depending on the nature of the therapy that is to be performed. Pulses of sound may be delivered to the ears isochronically at a single pitch, or two different pitches may be used simultaneously at different ears, to create pulses at the same rate as the difference in frequency between the pitches (i.e., as binaural beats). The method itself may be used therapeutically to promote relaxation and anxiety reduction in a mammalian subject, as has been described in detail herein, or for such other uses as inducing a meditative state in a mammalian subject or priming a subject for hypnosis. Other features and benefits will be understood by persons of skill as a result of the disclosure herein, as well as from the claims and drawings.

We claim:

1. A method of treating a mammalian subject, comprising the steps of:
measuring a wave-form produced by the electrical activity of the subject's brain;
analyzing said wave-form to determine changes in its frequency; and
delivering to the subject audio and visual stimulation, each at a rate selected to vary said frequency of said wave-form to a desired frequency of said wave-form.

2. The method of claim 1, wherein said step of delivering audio and visual stimulation includes the step of delivering pulses of light to one of the subject's eyes.

3. The method of claim 2, wherein said step of delivering pulses of light comprises the step of delivering pulses of light separately to the right and left visual fields of one of the subject's eyes.

4. The method of claim 1, wherein said step of delivering audio and visual stimulation includes the step of delivering pulses of sound to one of the subject's ears.

5. The method of claim 1, wherein said step of delivering audio and visual stimulation to the subject includes the steps of delivering pulses of light to one of the subject's eyes and delivering pulses of sound to one of the subject's ears.

6. The method of claim 5, wherein said steps of delivering pulses of light and delivering pulses of sound are performed simultaneously.

7. The method of claim 6, wherein said steps of delivering pulses of light and delivering pulses of sound are performed so that said pulses of light are synchronized with said pulses of sound.

8. The method of claim 1, further including the step of selecting said rate to correspond to a rate known to induce alpha wave-forms in the subject.

9. The method of claim 8, wherein said method includes the step of monitoring said changes in frequency, and said selecting step is performed in response to said changes in frequency of the wave form.

10. The method of claim 9, wherein said selecting step is performed by the subject.

11. The method of claim 9, wherein said selecting step is performed by a person other than the subject.

12. The method of claim 9, wherein said selecting step is performed automatically by a feedback mechanism.

13. A system for treating a mammalian subject, comprising:
a brain wave measuring device for measuring a wave-form representative of the electrical activity of the subject's brain;
an analyzer for determining changes in the frequency of said wave-form;
a light-producing element for providing pulses of light to the right visual field of the subject at a first rate and to the left visual field of the subject at a second rate;
a sound transducer for providing pulses of audible sounds to one of the subject's ears at a third rate; and
a control system for varying said first, second and third rates in response to said changes in the frequency of said wave-forms.

14. The system of claim 13, wherein said measuring device and said analyzer are included within an electroencephalograph having an electrode.

15. The system of claim 13, wherein said control system comprises a means for incorporating said frequency of said wave-form, said light-producing element and said sound transducer in a feedback loop.

16. The system of claim 15, wherein said control system comprises a means for varying said first, second or third rates through intervention by the subject.

17. The system of claim 15, wherein said control system comprises a means for varying said first, second or third rates through intervention by a therapist.

18. The system of claim 15, wherein said control system comprises a means for automatically varying said first, second or third rates without intervention by the subject or a therapist.
